(19) United States
(12) Patent Application Publication
(10) Pub. No.: US 2006/0111417 A1
(43) Pub. Date: May 25, 2006

(54) Amorphous telmisartan

(76) Inventors: Purandhar Koilkonda, Hyderabad (IN); Amarnath Reddy Lekkala, Hyderabad (IN); China Mala Kondaiah Golla, Hyderabad (IN); Santhi Reddy Ganji, Guntur (IN); Ramesh Babu Konda, Karimnagar (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/285,844

(22) Filed: Nov. 23, 2005

Related U.S. Application Data
(60) Provisional application No. 60/630,284, filed on Nov. 23, 2004.

(51) Int. Cl. C07D 403/06; A61K 31/4184

(52) U.S. Cl. 514/394; 548/305.7

(57) ABSTRACT

Amorphous telmisartan and combinations of amorphous telmisartan with one or more pharmaceutical carriers.

### CROSS-REFERENCE TO RELATED APPLICATION

This application is a nonprovisional filing of copending U.S. Provisional Application No. 60/630,284, filed Nov. 23, 2004, the entire disclosure of which is incorporated herein by this reference.

### INTRODUCTION TO THE INVENTION

The present invention relates to an amorphous form of telmisartan and a process for the preparation thereof.

Telmisartan has the chemical names 4′-[[(2-n-propyl-4-methyl-6-(1-methylbenzimidazol-2-yl)-benzimidazol-1-yl]-methyl]-biphenyl-2-carboxylic acid, or 4′-[(1,4′-dimethyl-2′-propyl [2,6′-bi-1H-benzimidazol]-1′-yl)methyl]-[1,1′-biphenyl]-2-carboxylic acid, and has the structural formula as in Formula I.

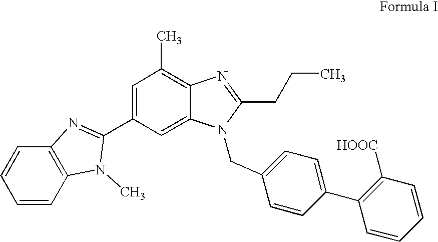

Telmisartan is an angiotensin-II receptor antagonist, useful in the treatment of hypertensive diseases, heart diseases, heart strokes and bladder diseases. It is commercially available in the form of tablets sold under the brand name MICARDIS™.

U.S. Pat. No. 5,591,762 discloses telmisartan and its pharmaceutically acceptable salts, along with its pharmaceutical compositions.

U.S. Pat. No. 6,358,986 discloses polymorphs of telmisartan, particularly polymorphic form B, mixture of the polymorphs, process for preparing telmisartan form B and the use thereof for preparing pharmaceutical compositions.

It is known that different polymorphic forms of the same drug may have substantial differences in certain pharmaceutically important properties. The amorphous form of a drug may exhibit different dissolution characteristics and in some case different bioavailability patterns compared to crystalline forms.

Further, amorphous and crystalline forms of a drug may have different handling properties, dissolution rates, solubility, and stability, Access to a choice between the amorphous or crystalline forms of a drug is desirable for different applications. Furthermore, different physical forms may have different particle size, hardness and glass transition temperatures. Amorphous materials do not exhibit the three-dimensional long-range order found in crystalline materials, but are structurally more similar to liquids where the arrangement of molecules is random. Amorphous solids do not give a definitive x-ray diffraction pattern (XRD). In addition, amorphous solids do not give rise to a melting point and tend to liquefy at some point beyond the glass transition temperature. Because amorphous solids do not have lattice energy, they usually dissolve in a solvent more rapidly and consequently may provide enhanced bioavailability characteristics such as a higher rate and extent of absorption of the compound from the gastrointestinal tract. Also, amorphous forms of a drug may offer significant advantages over crystalline forms of the same drug in solid dosage form manufacture process such as compressibility, economically or environmentally suitable solvents or process, or higher purity or yield of the desired product.

Regulatory authorities desire to have all possible polymorphic forms of a new drug substance identified prior to approval of a product containing the drug. However, as is well known in the art, the existence of polymorphic forms of any given compound cannot be predicted, and there is no standard procedure for proceeding to make a previously unknown polymorphic form. Even after a polymorph has been identified, there is no possibility of predicting whether any additional forms will ever be discovered. This situation has been the subject of recent articles, including A. Goho, “Tricky Business,” Science News, Vol. 166, No. 8, pages 122-123 (August 2004).

Consequently, it would be a significant contribution to the art to provide an amorphous form of telmisartan, haveing increased solubility, and methods of preparation, pharmaceutical formulations, and methods of use therefor.

### BRIEF DESCRIPTION OF THE DRAWINGS

FIG. 1 is an X-ray powder diffraction (“XRPD”) pattern for amorphous telmisartan.

### SUMMARY OF THE INVENTION

The invention relates to amorphous telmisartan.

The invention further relates to a process for the preparation of amorphous telmisartan.

The invention also relates to a composition that comprises telmisartan in a solid form, wherein at least 80% by weight of the solid telmisartan is an amorphous form of telmisartan.

In another embodiment, the invention provides a pharmaceutical composition that contains an amorphous form of telmisartan and one or more pharmaceutically acceptable carriers.

The invention further provides a process for preparing amorphous telmisartan, comprising removing solvent from a solution comprising telmisartan.

### DETAILED DESCRIPTION

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

The phrase “amorphous telmisartan” is intended to include any amorphous form of telmisartan including but not limited to amorphous telmisartan, an amorphous solid dispersion of telmisartan, and amorphous combinations of telmisartan with pharmaceutically acceptable carriers or crystallization inhibitors.

Unless stated to the contrary, any use of the words such as “including,” “containing,” “comprising,” “having” and the like, means “including without limitation” and shall not be construed to limit any general statement that it follows to the specific or similar items or matters immediately following it. Embodiments of the invention are not mutually exclusive, but may be implemented in various combinations. The described embodiments of the invention and the disclosed examples are given for the purpose of illustration rather than limitation of the invention as set forth in the appended claims. For purposes of the present invention, the following terms are defined below.

A “compound” is a chemical substance that includes molecules of the same chemical structure.

“Pharmaceutically acceptable” means that which is useful in preparing a pharmaceutical composition that is generally non-toxic and is not biologically undesirable and includes that which is acceptable for veterinary use and/or human pharmaceutical use.

The term “composition” includes, but is not limited to, a powder, a suspension, an emulsion and/or mixtures thereof. The term composition is intended to encompass a product containing the specified ingredients in the specified amounts, as well as any product, which results, directly or indirectly, from combination of the specified ingredients in the specified amounts. A “composition” may contain a single compound or a mixture of compounds.

The term “pharmaceutical composition” is intended to encompass a product comprising the active ingredient(s) and pharmaceutically acceptable excipients, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing the active ingredient, any additional active ingredient(s), and pharmaceutically acceptable excipients.

The term “excipient” means a component of a pharmaceutical product that is not the active ingredient, such as filler, diluent, carrier, and so on. The excipients that are useful in preparing a pharmaceutical composition are preferably generally safe, non-toxic and neither biologically nor otherwise undesirable, and are acceptable for veterinary use as well as human pharmaceutical use. A “pharmaceutically acceptable excipient” as used in the specification and claims includes either one, or more than one, such excipient.

The term “isolating” is used to indicate separation of the compound being isolated regardless of the purity of the isolated compound from any unwanted substance, which presents with the compound as a mixture. Thus, degree of the purity of the isolated or separated compound does not affect the status of “isolating.”

It has been found that obtaining an amorphous solid form of telmisartan is not a simple matter. Thus, in accordance with an aspect of the invention a particular process for preparation of the amorphous form of telmisartan is also provided. The process comprises: removing the solvent from a solution comprising telmisartan, such as by spray drying or agitated thin film drying techniques, to obtain a solid residue, which contains the amorphous form of telmisartan.

The solution of telmisartan can be prepared by dissolving any form of telmisartan, such as a crystalline form, an amorphous form, a solvate, or a hydrate, in a suitable organic solvent, or an existing solution can be obtained from a previous processing step, such as a final step in the synthesis of the compound, and subjected to the process for forming amorphous telmisartan.

Solvents that can be used for the process include any solvent or mixture of solvents, in which the required components are soluble. Examples include: straight or branched chain alcohols such as methanol, ethanol, n-propanol, isopropanol, butanol and the like; halogenated hydrocarbons such as dichloromethane, carbon tetrachloride, chloroform and the like; ketones such as acetone, methyl ethyl ketone, 2-pentanone and the like; ethers such as tetrahydrofuran, diethyl ether, diisopropyl ether, methyl t-butyl ether and the like; and mixtures thereof in any proportions.

The dissolution temperatures can range from about 10 to 45° C., or about 20 to 40° C., or at the reflux temperature of the solvent used.

The concentration of the solution of telmisartan before solvent removal can range from about 1% to 20% w/v, or about 5% to 12%, or about 8% to 11% w/v.

Removal of the solvent is accomplished by using any method of drying, including spray drying, rotational evaporation (such as using a Buchi Rotavapor), agitated thin film drying (ATFD), spin-flash drying, fluid-bed drying, lyophilization, or other techniques known in art.

In one embodiment, vertical agitated thin-film drying (or evaporation) is used for drying of the telmisartan solution in the organic solvents of the invention. ATFD involves separating the volatile component using indirect heat transfer coupled with mechanical agitation of the flowing film under controlled conditions. In vertical ATFD (ATFD-V), the solution to be dried is fed from the top into a cylindrical space between a centered rotary agitator and an external heating jacket to form a thin film. The rotation of the rotor agitates the solution flowing downward while the heating jacket heats it.

In another aspect of the embodiment, spray-drying is used for drying the solution of telmisartan. The solution of telmisartan is sprayed into the spray drier at a flow rate typically ranging from 10 to 300 ml/hr, or 40 to 210 ml/hr. The air inlet temperature to the spray drier can range from 70° C. to 150° C., or from 85° C. to 110° C., and the outlet air temperature used can range from 30° C. to 80° C. Of course, specific conditions will vary somewhat for spray drying using different equipment configurations. The solid residue obtained after the solvent removal is isolated and, if desired, can be dried further using conventional methods. The advantages of the process include simplicity, eco-friendliness and suitability for commercial use.

Amorphous telmisartan can also be prepared in the presence of one or more pharmaceutically acceptable carriers, including those that act as crystallization inhibitors. Without being bound by any particular theory, it is expected that the drug would be distributed within the carrier at a molecular level, resulting in an amorphous material that is a molecular dispersion. Thus, for such a material the energy required for breaking down the crystal structure to bring the drug into solution is reduced, thereby resulting in enhanced solubility, more rapid dissolution, or both. Further, such materials can also act as crystallization inhibitors to prevent the conversion of the amorphous form of telmisartan into a crystalline form, thus resulting in enhanced stability of the compound at conventional storage temperatures.

Such amorphous telmisartan along with a carrier can be prepared by dissolving telmisartan in a organic solvent, dissolving a pharmaceutically acceptable carrier in a second organic solvent, combining the two solutions, and subjecting the combination to evaporation by any conventional means of drying. The organic solvent used for dissolving the telmisartan and the pharmaceutically acceptable carrier can be the same or different solvents can be used. In both of the above mentioned cases, in some instances it may be required to raise the temperature to enhance the dissolution of the telmisartan and the pharmaceutically acceptable carrier. Any temperature is acceptable as long as this does not cause degradation of the product. If both the amorphous telmisartan and the carrier have the required solubility, under approximately the same conditions, in the same solvent, then they can be used to form a single solution in a single solvent.

Pharmaceutically acceptable carriers that can be used for the preparation of amorphous telmisartan include, but are not limited to, pharmaceutical hydrophilic carriers such as polyvinylpyrrolidone (homopolymers or copolymers of N-vinylpyrrolidone), gums, cellulose derivatives (including hydroxypropyl methylcellulose, hydroxypropyl cellulose and others), cyclodextrins, gelatins, hypromellose phthalate, sugars, polyhydric alcohols, polyethylene glycol, polyethylene oxides, polyoxyethylene derivatives, polyvinyl alcohol, propylene glycol derivatives and the like. The use of mixtures of more than one of the pharmaceutical carriers to provide desired release profiles or for the enhancement of stability is within the scope of this invention. Also, all viscosity grades, molecular weights, commercially available products, their copolymers, mixtures are all within the scope of this invention without limitation.

Of course, the rates of drying, the concentrations of the respective solutions, the ratios of the telmisartan and the pharmaceutically acceptable carrier to achieve a stable amorphous product can be defined by a person skilled in the art through simple experimentation, and are all included herein without limitation.

In another aspect, the present invention provides a pharmaceutical composition containing the amorphous form of telmisartan and one or more pharmaceutical excipients. The pharmaceutical composition may be prepared by uniformly admixing the active ingredient with liquid or solid excipients and then shaping the product into the desired form. The pharmaceutical compositions may be in the form of suspensions, solutions, elixirs, aerosols, or solid dosage forms. Because of their ease of administration, tablets and capsules represent a more advantageous oral dosage unit form, in which case solid pharmaceutical excipients are employed.

Pharmaceutical excipients that can be used for the preparation of pharmaceutical compositions include, but are not limited to, hydrophilic polymers such as polyvinylpyrrolidone (homopolymers or copolymers of N-vinylpyrrolidone), gums, cellulose derivatives (including hydroxypropyl methylcellulose, hydroxypropyl cellulose and others), cyclodextrins, gelatins, hypromellose phthalate, sugars, polyhydric alcohols, polyethylene glycol, polyethylene oxides, polyoxyethylene derivatives, polyvinyl alcohol, propylene glycol derivatives and the like. The use of mixtures of more than one of the pharmaceutical carriers to provide desired release profiles or for the enhancement of stability is within the scope of this invention. Also, all viscosity grades, molecular weights, commercially available products, their copolymers, mixtures are all within the scope of this invention without limitation. Those having skill in the pharmaceutical formulation art are well aware of numerous suitable excipients, and useful procedures for making pharmaceutical compositions.

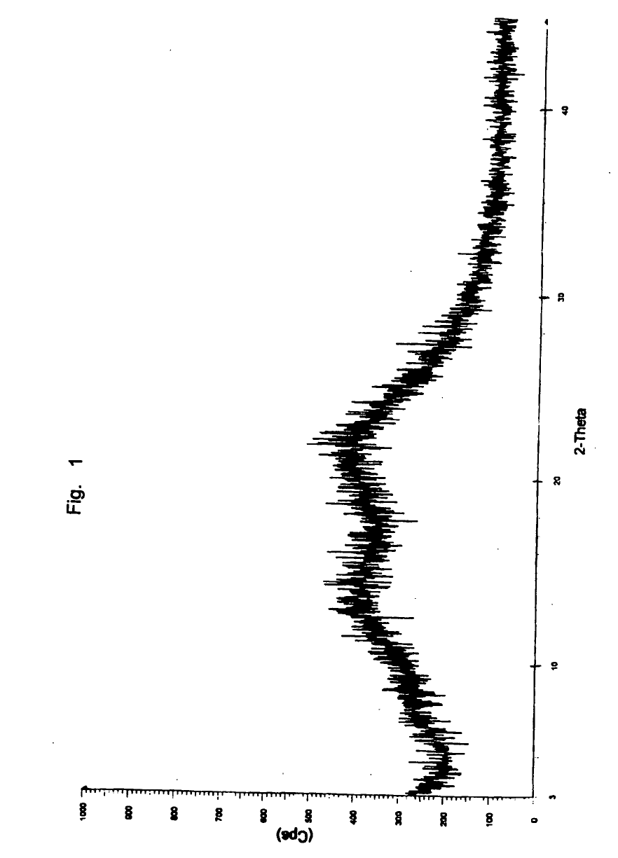

The XRPD of the amorphous telmisartan was measured on a Bruker Axe, DS Advance Powder X-ray Powder Diffractometer using a Cu K alpha-1 radiation source. Amorphous telmisartan is characterized by its XRPD pattern substantially in accordance with FIG. 1, where the vertical axis is intensity and the horizontal axis is the 2θ angle, in degrees.

An embodiment of the invention provides telmisartan having at least about 80 percent by weight, or at least about 90 percent by weight, or at least about 95 percent by weight, of amorphous telmisartan. A further embodiment provides telmisartan in combination with a pharmaceutical carrier, wherein at least about 80 percent by weight, or at least about 90 percent by weight, or at least about 95 percent by weight, of the contained telmisartan is amorphous.

The invention is further described by reference to the following examples, which set forth in detail certain aspects and embodiments of the preparation of compounds and compositions of the present invention. It will be apparent to those skilled in the art, that many modifications, both to materials and methods, can be practiced without departing from the purpose and intent of this invention. The examples that follow are not intended to limit the scope of the invention as described hereinabove or as claimed below.

### EXAMPLE 1

Preparation of Amorphous Telmisartan

2 g of crystalline telmisartan was taken into a round bottom flask to which 250 ml of dichloromethane was added. The solution was filtered through paper and cloth and washed with 20 ml of dichloromethane. The filtrate was distilled to dryness under reduced pressure at about 40 to 45° C. for about 45 minutes until the solvent was completely removed. The solid thus obtained was scraped from the flask and dried under high vacuum at a temperature of about 50° C. for about 6 hours to get 1.5 g of the desired amorphous form of telmisartan.

### EXAMPLE 2

Preparation of Amorphous Telmisartan by Spray Drying

5 g of telmisartan was dissolved in 700 ml of dichloromethane in a round bottom flask. The solution was filtered through paper and cloth and was then spray dried at 40 to 45° C. for about 90 minutes until the solvent was completely removed. The solid thus obtained was collected and dried under high vacuum at a temperature of 42° C. for about 5 hours to get 0.6 g of the desired amorphous form of telmisartan.

### EXAMPLE 3

Preparation of Amorphous Telmisartan by Agitated Thin Film Drying

5 g of telmisartan and 500 ml of dichloromethane are charged into a clean and dry round bottom flask and stirred for about 30 minutes. The solution is filtered through paper and cloth. The filtrate is subjected to agitated thin film drying with a feed rate of about 5 L/hour, under a reduced pressure of about 5-20 torr and a jacket temperature of about 35-40° C. The solid obtained is further dried at about 30-40° C. under reduced pressure of about 50-100 mbar for about 5-6 hours to afford 3 g of the title compound.

### EXAMPLE 4

Preparation of an Amorphous Telmisartan Composition by Agitated Thin Film Drying

5 g of telmisartan, 5 g of polyvinylpyrolidone (PVP K-30), and 750 ml of dichloromethane are charged into a clean and dry round bottom flask and stirred for about 30 minutes. The solution is filtered through paper and cloth. The filtrate is subjected to agitated thin film drying with a feed rate of about 5 L/hour, under a reduced pressure of about 5-20 torr and a jacket temperature of about 35-40° C. The solid obtained is further dried at about 30-40° C. under reduced pressure of about 50-100 mbar for about 5-6 hours to afford 6 g of the title composition.

We claim:

1. Amorphous telmisartan.

2. The amorphous telmisartan of claim 1, in combination with a pharmaceutically acceptable carrier.

3. The amorphous telmisartan of claim 1, combined with a pharmaceutically acceptable carrier to form a molecular dispersion.

4. The amorphous telmisartan of claim 1, combined with a pharmaceutically acceptable carrier to form an amorphous molecular dispersion.

5. A pharmaceutical composition prepared by combining amorphous telmisartan of claim 1 and at least one excipient.

6. A pharmaceutical composition prepared by combining the amorphous telmisartan molecular dispersion of claim 2 and at least one excipient.

7. A pharmaceutical composition prepared by combining the amorphous telmisartan molecular dispersion of claim 3 and at least one excipient.

8. A process for preparing amorphous telmisartan, comprising removing solvent from a solution comprising telmisartan to form a solid amorphous residue comprising telmisartan.

9. The process of claim 8, wherein the solution further comprises a pharmaceutically acceptable carrier.

10. The process of claim 8, wherein a solvent comprises a straight or branched chain alcohol, a halogenated hydrocarbon, a ketone, an ether, or a mixture of any two or more thereof.

11. The process of claim 8, wherein least about 80 percent by weight of telmisartan in the residue is amorphous.

12. The process of claim 8, wherein at least about 90 percent by weight of telmisartan in the residue is amorphous.

13. The process of claim 8, wherein t at least about 95 percent by weight of telmisartan in the residue is amorphous.

14. A process for preparing a dispersion comprising amorphous telmisartan, comprising dissolving telmisartan in a solvent, dissolving a pharmaceutical carrier in a solvent, combining solutions of telmisartan and a pharmaceutical carrier, and removing solvent to form a solid.

15. The process of claim 14, wherein a solvent for dissolving telmisartan and a solvent for dissolving a pharmaceutical carrier are different.

16. The process of claim 14, wherein a solvent for dissolving telmisartan comprises a straight or branched chain alcohol, a halogenated hydrocarbon, a ketone, an ether, or a mixture of any two or more thereof.

17. The process of claim 14, wherein at least about 80 percent by weight of telmisartan in the solid is amorphous.

18. The process of claim 14, wherein at least about 90 percent by weight of telmisartan in the solid is amorphous.

19. The process of claim 14, wherein at least about 95 percent by weight of telmisartan in the solid is amorphous.
